(19) United States
(12) Patent Application Publication
(10) Pub. No.: US 2004/0143240 A1
(43) Pub. Date: Jul. 22, 2004

(54) Adjustable length catheter

(76) Inventors: Joseph R. Armstrong, Flagstaff, AZ (US); Edward H. Cully, Flagstaff, AZ (US); John R. Daugherty, Flagstaff, AZ (US); Eric G. Johnson, Flagstaff, AZ (US); David R. King, Wilmington, DE (US); Mark J. Ulm, Flagstaff, AZ (US); Michael J. Vonesh, Flagstaff, AZ (US)

Correspondence Address: W. L. Gore & Associates, Inc., 551 Paper Mill Road, P.O. Box 9206, Newark, DE, 19714-9206, US

(21) Appl. No.: 10/346,977

(22) Filed: Jan. 17, 2003

(51) Int. Cl. A61M 25/01

(52) U.S. Cl. 604/528; 604/103.04

(57) ABSTRACT

A catheter provided with an adjustable length guidewire catheter lumen, located proximal of a therapeutic device or agent positioned at the distal end of the catheter. The length of the adjustable length guidewire catheter lumen is controlled by the physician, allowing the benefits of both over-the-wire and rapid exchange systems to be provided in one catheter. The adjustable length is provided with a thin-walled tube that corrugates under axial compression. The tube may optionally be pre-corrugated or may be allowed to corrugate non-uniformly under the axial compression. The catheter length may change by, for example, over 100% of its original length between full axial compression and full axial extension.

### FIELD OF THE INVENTION

The present invention relates to the field of catheters for use with guidewires, and more particularly to such catheters intended for the delivery of a therapeutic agent or device.

### BACKGROUND OF THE INVENTION

A variety of different therapies can be delivered within the human body by catheter devices. Therapeutic devices such as dilation balloons, stents, and embolic filters, and therapeutic agents such as drugs and radiation sources, may be positioned at or near the distal end of the catheter for delivery to a desired site within the body. The proximal end of the catheter is considered to be the end that remains outside of the body, manipulated by the medical practitioner.

To aid in positioning of the distal end of the catheter within the body, typically the distal end of a guidewire is first navigated to the treatment area. After the guidewire has been positioned, the wire can then be used to guide the distal end of the catheter into place. Additionally, a guide catheter may be used to further facilitate the positioning of the guidewire and/or delivery catheter. The interaction between the guidewire and the catheter is critical, as the physician needs to easily track the distal end of the catheter along the path of the guidewire. A number of interaction issues can arise, including but not limited to, having to use more than one person, having to use a long wire, having the advancement of the catheter affect the position of the wire, having the catheter not able to track the wire through tortuous anatomy, having excessive friction between the catheter and the wire, and having a difference between the amount of axial motion applied to the proximal end of the catheter and the amount of axial movement at the distal end of the catheter.

In various attempts to address these issues, a number of catheter designs have been introduced that have defined the interaction between the guidewire and the catheter. Two of the primary applications of catheter systems are percutanous transluminal coronary angioplasty (PTCA) and coronary stent delivery. Two main types of catheter designs, over-the-wire (OTW) and rapid-exchange (RX), dominate these applications. Each of these designs has its advantages and disadvantages. OTW catheters track over their entire length on a guidewire, which allows them to follow the wire easily and allows the direct transmission of longitudinal force over the guidewire. Additionally, these catheters allow for guidewires to be exchanged once the catheter has been advanced into position, which may be desirable when a different guidewire attributes (e.g., tip curvature or radiopaque markers) are needed. However, these systems require the use of a long guidewire (e.g., 300 cm in length) and cannot be effectively operated by one person.

RX catheters typically use shorter guidewires (e.g., 180 cm in length) which allow the catheter to be operated by a single physician. The physician is able to hold the guide catheter and guidewire with one hand while using his other hand to advance or retract the catheter along the guidewire. However, because the entire length of the RX catheter does not slide over the guidewire, the direct transmission of longitudinal force along the path of the guidewire may be compromised, and wire exchange can not be performed once the proximal catheter guidewire port is advanced into the patient.

Among various catheter designs intended for stent delivery is a system taught by U.S. Pat. No. 5,534,007 to St. Germain et al. This system includes a tubular exterior sleeve with an adjustable length section that, under axial compression, shortens via corrugations to cause another sleeve at the distal end of the catheter to be withdrawn in a proximal direction, releasing the stent. The overall length of the catheter remains the same during the axial compression of the exterior sleeve, and in particular, the length of the guidewire lumen is not adjustable.

### SUMMARY OF THE INVENTION

The present invention relates to a catheter provided with an adjustable length guidewire catheter lumen, proximal of a therapeutic device or agent positioned at the distal end of the catheter. The length of the adjustable length lumen is controlled by the physician, allowing the benefits of both OTW and RX systems to be provided in one catheter.

The adjustable length catheter guidewire lumen is the conduit, or catheter, or tube, or space, that contains the guidewire or provides a space for the passage of a guidewire therethrough. The space is adjustable in length, as will be further described.

By adjustable length is meant that the length of the adjustable length guidewire catheter lumen may be changed by the application of easily-applied manual axial force. In its axially extended or fully lengthened state, the adjustable length guidewire catheter lumen is at least 10% longer than when in the axially compressed, fully shortened state. More preferably, the adjustable length guidewire catheter lumen is adjustable by an amount of at least about 20%, or 30%, or 40%, or 50%, or 75%, or 100%, or 200%, or 400%, or 1000%, or 2000%.

The adjustable length guidewire catheter lumen is adjustable in length by virtue of being scrunchable. This means that this tubular component is easily shortened in length under axial force, without telescoping as by the successive sliding of overlapped concentric tubular sections. Various means of providing a scrunchable tube for use as the adjustable length guidewire catheter lumen include the provision of corrugations (i.e., wrinkles, or accordion pleats or folds), or by the use of a porous tube that compresses axially by reduction in total void space. These are further described below.

The catheter assembly of the present invention may include a fixed length guidewire catheter that is coextensive with the adjustable length catheter guidewire lumen, meaning that together the guidewire catheter and the adjustable length catheter guidewire lumen form a continuous passageway for a guidewire. Preferably, neither the guidewire catheter nor the adjustable length guidewire catheter lumen include any apertures or ports through the wall of either one that might be used to pass a guidewire through to the exterior of either, or be used for any other functional purpose.

The present invention addresses a number of the shortcomings of OTW and RX systems. It allows the full length of the catheter within the patient's body to be fully supported by a guidewire, and it also allows the physician the convenience of operating the catheter system independently (without assistance) while using a short guidewire. By incorporating a thin-walled (e.g., less than about 0.20 mm wall thickness, and more preferably less than about 0.10 mm) adjustable length component into the catheter, the positive attributes of both OTW and RX systems may be made available in a single catheter system.

Additionally, the adjustable length guidewire catheter lumen is particularly flexible. The excellent flexibility results from having the guidewire and other tubes (i.e. the inflation lumen) adjacently oriented and in substantially parallel, collateral relationship, providing greater flexibility than inherently stiffer coaxial constructions. Flexibility is enhanced because a conventional fixed length, relatively stiff guidewire catheter is not required. The distal tip portion of the catheter, including any distally positioned therapeutic device (e.g., a balloon), is preferentially less flexible than the adjustable length guidewire catheter lumen, and accordingly is provided with a less flexible coaxial construction.

For purposes of the present invention, collateral relationship of the adjustable length guidewire catheter lumen with other components of the catheter such as the inflation lumen, means that the adjustable length guidewire catheter lumen is substantially parallel to the other component and may consequently, also be coaxial with the other component.

Also, with the distal portion of the catheter advanced into position, the physician may chose to change out the guidewire for an alternative guidewire with, for example, different tip flexibility or different radiopaque markers. By tapering the distal connection of the adjustable length guidewire catheter lumen into the coaxial construction of the distal tip portion (e.g., a funneling connection), a guidewire may be advanced from the proximal guidewire port and be directed through the catheter out the distal guidewire port.

Further, the catheter system may be provided with a small, proximal three exit port fitting, which is in effect a y-fitting in combination with a hub component that allows attachment of an inflation syringe to dilate a distally-positioned balloon. By placing this three exit port fitting adjacent to the proximal end of the guide catheter, or adjacent to a hemostasis valve attached to the guide catheter, the physician can hold both the guide catheter and proximal three exit port fitting, and control all of the functions of the catheter from one location. Additionally, the use of a looped inflation lumen (described herein below) minimizes the risk of contamination of the portion of the catheter outside of the patient.

In a preferred embodiment, the proximal three exit port fitting may be fixed by the physician to a set location along the inflation lumen of the catheter. One technique of fixing the axial position of the proximal three exit port fitting is by the use of a hemostasis valve that incorporates a compressible elastomeric o-ring. By the application of a compressive force to the o-ring, the position of the three exit port fitting relative to the inflation lumen can be fixed. By fixing the three exit port fitting to the inflation lumen, with the adjustable length guidewire catheter lumen in its fully compressed (i.e., fully shortened) configuration, the entire catheter assembly can be easily and quickly removed from the proximal end of the guidewire.

Additionally, the y-fitting may be designed such that it cannot rotate around the inflation lumen, thereby preventing twisting and binding of the wire. One suitable technique involves the provision of an inflation lumen with a ‘D’ shaped cross section and a hub that incorporates a corresponding flat surface to prevent rotation relative to the inflation lumen.

Alternatively, the system of the present invention may be provided with lumens on the fixed-length portions of the catheter that have conventional circular transverse cross sections with components in coaxial relationship. These circular cross sections result in catheters with similar flexibility when bent in any direction.

By supporting the full length of the catheter between the distal tip portion (including the balloon) and the y-fitting or three exit port fitting (where the physician controls advancement and retraction of the catheter by pushing or pulling a pushable element such as the inflation lumen) with the adjustable length guidewire catheter lumen, stiff metal hypotubes typically required for conventional PTCA catheter inflation lumens are not necessary. Other materials, for example thermoplastics or thermoset plastics with or without braided or coil reinforcement, may be used.

To facilitate threading of a guidewire through the catheter, fixed length tubes may be used. These tubes are preferably thin-walled (e.g., less than about 0.2 mm wall thickness) and may either be permanently fixed to the catheter at one end of the fixed length tube, or may be made to be removable after the guidewire has been threaded through the catheter. If this threading tube is fixed, it is preferably attached to the sliding hub at its proximal end. At its distal end, it can slide partially or fully into or through the fixed length portion of the distal portion of the guidewire lumen. This is preferable because once the adjustable length lumen is extended, this threading tube separates from the distal portion of the catheter, thereby avoiding adversely affecting flexibility. After advancing the catheter, shortening of the adjustable length catheter guidewire lumen causes the distal end of the threading tube to track back over the guidewire and finally into or through the fixed length portion of the distal portion of the guidewire lumen.

Because the catheter is fully supported by containment of the guidewire, including the portion of the length of the guidewire within the adjustable length guidewire catheter lumen distal of the slideable y-fitting, the distal fixed length section of guidewire lumen (including the balloon and the catheter distal tip) can be very short. With the adjustable length lumen fully compressed (shortened), the y-fitting is very close to the distal tip of the catheter. This short distance only requires a short length of the proximal end of the guidewire wire to be exposed outside of the patient. This short threading length facilitates fast threading and removal of the catheter from the wire.

Suitable materials for the adjustable length lumen include expanded polytetrafluoroethylene (ePTFE), polyethylene terephthalate (PET), polyamide, or other thermoplastic or thermoset polymers, or other such relatively inelastic materials. Alternatively, an elastomeric material may be used for the adjustable length lumen, which materials elongate by the application of an extending axial force. The term “elastomeric” is intended to describe a condition whereby a polymer displays stretch and recovery properties similar to an elastomer, although not necessarily to the same degree of stretch and/or recovery.

The present invention addresses shortcomings of OTW and RX catheter systems. It allows the full length of the catheter within the patient's body to be fully supported by a guidewire, and allows the physician the convenience of operating the catheter system independently while using a short guidewire. By incorporating a thin-walled, adjustable length component of the catheter, the catheter system of the present invention provides the positive attributes of both OTW and RX systems.

### BRIEF DESCRIPTION OF THE DRAWINGS

FIG. 1A shows a longitudinal cross section of a catheter including a y-fitting and hub at the proximal end, and further having an adjustable length guidewire catheter lumen (shown in its axially compressed or shortened state).

FIG. 1B shows an enlargement of a portion of FIG. 1A.

FIG. 1C shows a longitudinal cross section of the catheter of FIG. 1A in its fully extended state.

FIG. 2 shows a longitudinal cross section of a basic embodiment of the catheter of the present invention, without a y-fitting but including a hub on the proximal end of the inflation lumen, an adjustable length guidewire catheter lumen (shown in its axially compressed or shortened state) located distal to the hub and a tubular slider for controlling the proximal end of the adjustable length lumen.

FIG. 3A shows a longitudinal cross section of a catheter similar to the catheter of FIG. 1A with the addition of a threading tube coaxial with the guidewire; the adjustable length guidewire catheter lumen is shown in its axially compressed or shortened state.

FIG. 3B shows an enlargement of a portion of FIG. 3A.

FIG. 4 shows a longitudinal cross section of a catheter having a y-fitting that includes a tubular extension at its distal end that protects the compressed adjustable length guidewire catheter lumen (shown in its axially compressed or shortened state) from the guidewire.

FIGS. 5A and 5B show alternative transverse cross sections of the adjustable length guidewire catheter lumen.

FIG. 6A shows a longitudinal cross section of a catheter guidewire having two different length portions, with a visual marker between them, that are separately coated with high-friction coatings that allow the operator to grip the catheter and any component within the coated portion, to prevent respective axial movement relative to the gripping hand.

FIGS. 6B and 6C show transverse cross sections of different portions of the catheter of FIG. 6A.

FIG. 7 shows a longitudinal cross section of a catheter having two adjustable length guidewire catheter lumens (the distal adjustable length guidewire catheter lumen shown in its axially compressed or shortened state while the proximal adjustable length guidewire catheter lumen is shown in its fully lengthened state) separated by a guidewire gripping component.

FIG. 8 shows a longitudinal cross section of a catheter having an adjustable length inflation lumen located outside of an adjustable length guidewire catheter lumen (with both of these lumens shown in their axially compressed or shortened states) wherein the length of both of these lumens is changed by the use of extending means such as a wire that may be pushed or pulled.

FIG. 9A shows a longitudinal cross section of a catheter having an adjustable length guidewire catheter lumen with guidewire clip.

FIG. 9B shows a transverse cross section of the guidewire clip of FIG. 9A in use on the catheter.

FIG. 10A shows a longitudinal cross section of a catheter having a puncturable guidewire lumen covering.

FIG. 10B shows a longitudinal cross section of the catheter of FIG. 10A in use with the catheter, the guidewire having punctured the puncturable guidewire lumen covering.

FIGS. 10C and 10D show transverse cross sections of the catheter of FIG. 10B with the guidewire within and without the puncturable section.

FIG. 10E shows a longitudinal cross section of a catheter that is a variation of the design shown in FIGS. 10A and 10B wherein the guidewire operates in a slot provided in the exterior wall of a lumen of the catheter.

FIGS. 10F, 10G and 10H show transverse cross sections taken at three different locations along the length of the catheter shown in FIG. 10E.

FIG. 11A shows a side view of a catheter having an adjustable length guidewire catheter lumen (shown in its axially compressed or shortened state) with the length of the inflation lumen that extends proximal to the y-fitting formed into a loop, with the hub of the inflation lumen affixed to the y-fitting to create a three exit port fitting.

FIG. 11B shows a side view of a catheter of FIG. 11A except that the adjustable length guidewire catheter lumen is now shown in its axially extended state, with the length of the inflation lumen that extends beyond the y-fitting formed into a loop that is reduced in length from the loop shown in FIG. 11A by the amount of the extension of the adjustable length guidewire catheter lumen.

FIGS. 12A and 12B show, respectively, longitudinal cross sections of a catheter having an external adjustable length guidewire catheter lumen, in axially compressed and fully extended states.

### DETAILED DESCRIPTION OF THE INVENTION

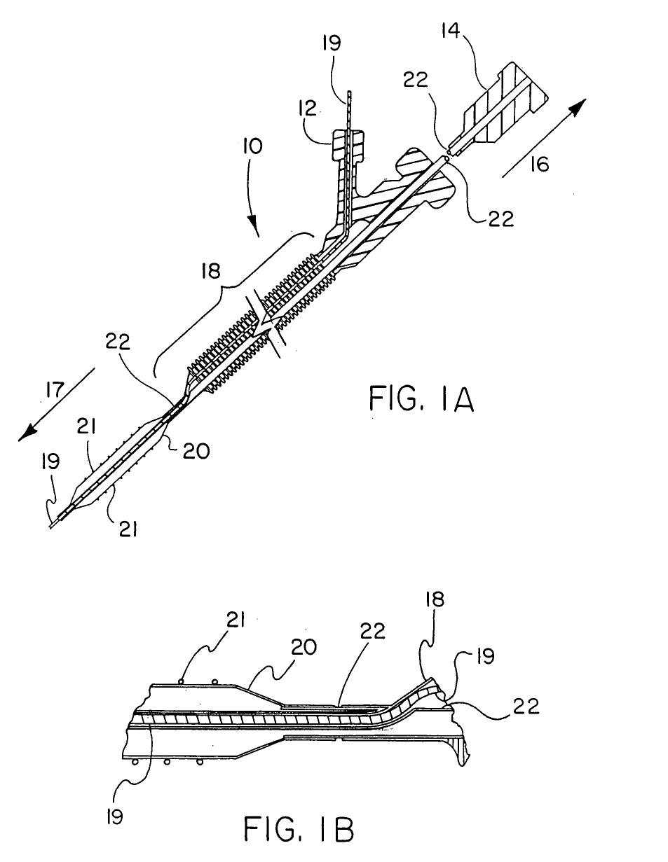

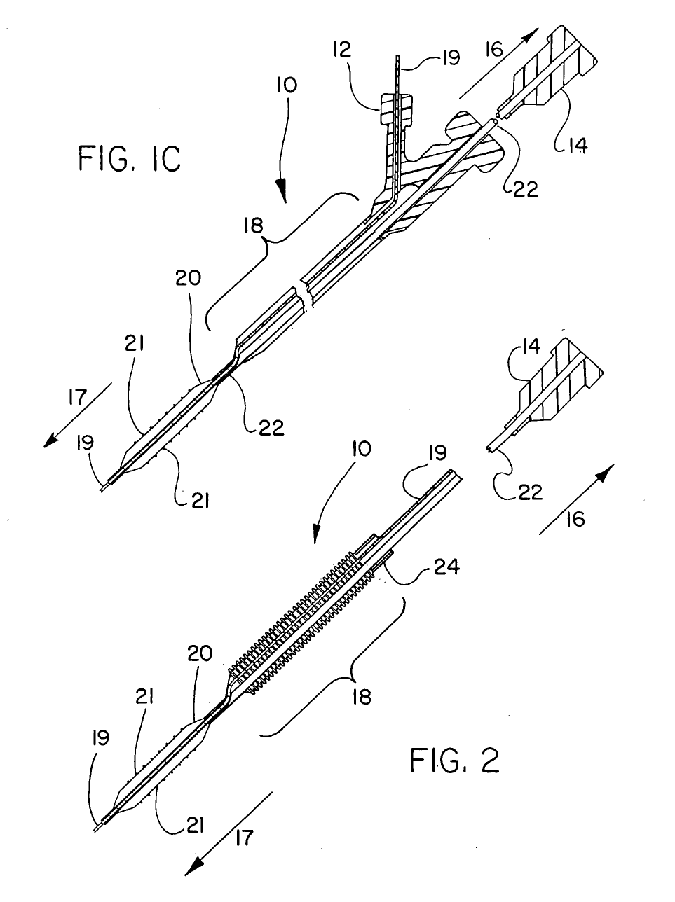

FIG. 1A shows a longitudinal cross section of a catheter 10 of the present invention, including a slideable y-fitting 12 and hub 14 at the proximal end 16, and further having an adjustable length guidewire catheter lumen 18, shown in its axially compressed or shortened state. An enlargement of the portion of catheter 10 located between the proximal end of balloon 20 and the distal end of the adjustable length guidewire catheter lumen 18 is described by the longitudinal cross section of FIG. 1B. FIG. 1C shows a longitudinal cross section of the same catheter 10 with the adjustable length guidewire catheter lumen 18 axially extended to its full length. The adjustable length section 18 is provided by the use of a thin tubular material that accommodates the axial compression by corrugations, elastomeric length recovery or by various other means. The catheter 10 is slideable along its full length on guidewire 19, and is supported by guidewire 19 along the entire length of this adjustable length section 18.

In practice, using a hemostasis valve such as a Touhy-Borst valve attached to the proximal end of a guide catheter, the physician can fix the axial position of the proximal end 16 of the adjustable length guidewire lumen 18, the y-arm 12, and the guidewire 19 as he advances the balloon 20 located at the distal end 17 into the patient's vasculature. For purposes of the present invention, many of the hemostasis valves referred to herein are used as mechanical gripping devices rather than as fluid control valves. This adjustable length guidewire catheter lumen 18 also allows the tubular portions of the catheter to remain essentially circular in transverse cross section, thereby avoiding the adverse effects that transversely asymmetrical components can have on the ability of the catheter 10 to follow the path of the guidewire 19.

Y-fitting 12 (preferably including hemostasis valves on both exit ports) is slideable along the length of the inflation lumen 22 in a conventional fashion. The pushable element, e.g., inflation lumen 22, is typically moved with respect to the y-fitting 12 by holding the y-fitting 12 in a fixed position with respect to the entrance of the catheter 10 into the patient's body, while pulling or pushing on the proximal end of inflation lumen 22, or on hub 14 located at the proximal end 16 of the inflation lumen 22. Pushing the pushable element (e.g., hub 14 or inflation lumen 22) causes inflation lumen 22 to slide distally through y-fitting 12, moving the distal end 17 of the catheter (including balloon 20, shown deflated, and optional stent 21) through the patient's body, simultaneously extending the adjustable length guidewire catheter lumen 18.

Adjustable length guidewire catheter lumen 18 may be made from a variety of thin, flexible polymer materials such as polyethylene, polypropylene, polyamide, polyethylene terephthalate, etc. Porous polymers, optionally provided with a thin, non-porous coating, may be advantageously used because of their excellent flexibility. Adjustable length guidewire catheter lumen 18 is preferably made from a porous expanded PTFE (ePTFE) film that has been provided with a porous or non-porous coating of a thermoplastic fluoropolymer, preferably fluorinated ethylene propylene (FEP). ePTFE films are generally made as taught by U.S. Pat. Nos. 3,953,566 and 4,187,390 to Gore. The construction of thin, helically-wrapped tubes from ePTFE films and FEP-coated ePTFE films, and the method of providing the coating onto the ePTFE films, are taught by U.S. Pat. No. 6,159,565 to Campbell et al.

In addition to the necessary axially compressible character of the adjustable length guidewire catheter lumen, adequate flexibility is ascertained in either of two fashions. First, an adequately flexible tube for use as the adjustable length guidewire catheter lumen will, when placed on a flat surface without any object occupying the luminal space and when fully axially extended, flatten under its own weight to the extent that its height (as measured vertically from the flat surface) is equal to 90% or less of its width. Alternatively, a length of suitable tubing is placed on a flat surface with the length parallel to that surface, again with the luminal space unoccupied and the tube fully axially extended. A 2 cm length of the tube is pushed over an edge of the flat surface so that it is no longer supported by that surface. If the tip (i.e., the lowest point of the very end edge of the tube) of that 2 cm length drops below the level of the flat surface by an amount of at least 1 mm, the tube is considered to be flexible.

The thin-walled tube is preferably made from an FEP-coated ePTFE film that has been cut into a tape (width, e.g., 12.7 mm) and helically wrapped on a mandrel with the FEP coating placed on the exterior of the wrapping. The helically wrapped tube is then placed into an oven for a suitable time (e.g., 8 minutes in an oven set at a temperature of 320° C.) to thermally bond the overlapped edges of the helical wrapping together, thereby forming a coherent tube. After removal from the oven and cooling, the resulting tube is removed from the mandrel and may be used as the adjustable length lumen component in the catheter of the present invention. The ends of this tube may be joined to the adjacent components by overlapping the tube end over the adjacent component and adhering the overlapped areas with an adhesive such as a cyanoacrylate (e.g., Loctite 401, Rocky Hill, Conn.) or an ultraviolet adhesive (e.g., Loctite 3311). Alternatively, the tube may be everted to orient the FEP-coating toward the lumen, and an adequate heat source may be used to melt-bond the FEP coating to catheter components such as metal hypotubes.

For use as the adjustable length lumen tubular component of a catheter, the ePTFE tube may be provided with corrugations (e.g, accordion pleats or folds) with various methods such as those taught by U.S. Pat. No. 3,105,492 to Jeckel and U.S. Pat. No. 6,016,848 to Egres, Jr. Alternatively, it is not required to provide the thin-walled tube with preformed corrugations as, during axial compression from the fully extended length to the shortened, fully compressed length, the tube will wrinkle and corrugate in a non-uniform but entirely suitable manner for use as the adjustable length lumen portion 18 of catheter 10. In another alternative, an elastomer may be used for the adjustable length portion 18 that would be in its relaxed state prior to loading over the guidewire and would extend into a tensioned condition when the distal end of the catheter is advanced.

Longitudinally extruded and expanded tubes of PTFE, that is, seamless ePTFE tubes, may be used in thinwall form as the adjustable length guidewire catheter lumen. Under axial compression, the interconnecting fibrils of the node-and-fibril microstructure of ePTFE will progressively bend and fold. This allows the tubular material to axially compress in a substantially uniform fashion, retaining the longitudinal uniformity of the tube wall (macroscopically), without corrugations. This bending of the fibrils within the microstructure of the wall of the ePTFE tube during axial compression is described in U.S. Pat. No. 4,877,661 to House et al. Longer mean fibril length tubes are preferred to maximize the compressible length, e.g., ePTFE tubes of about 50 micron or greater mean fibril length.

FIG. 2 shows a longitudinal cross section of a basic embodiment of catheter 10, without a y-fitting 12 but including a hub 14 on the proximal end 16 of the inflation lumen 22. A tubular slider 24 is used in place of y-fitting 12, distal to hub 14 for attachment and control of the proximal end of the adjustable length guidewire catheter lumen 18. Catheter 10 further includes an adjustable length guidewire catheter lumen 18 (shown in its axially compressed or shortened state). The tubular slider 24 may or may not allow the guidewire to exit the catheter. As shown, the tubular slider 24 is open to the exterior of the catheter, allowing the proximal end of the guidewire 19 to exit the catheter. Alternatively, there may be only a small clearance between the inner diameter of slider 24 and the outer diameter of the inflation lumen 22. By designing this slider 24 with two coaxial elastomeric o-rings that pinch the guidewire 19 between one another, once guidewire 19 is inserted and engaged into slider 24, the position of slider 24 can be used to control the position of the guidewire 19. Accordingly, the proximal end of guidewire 19 may remain fully within the catheter.

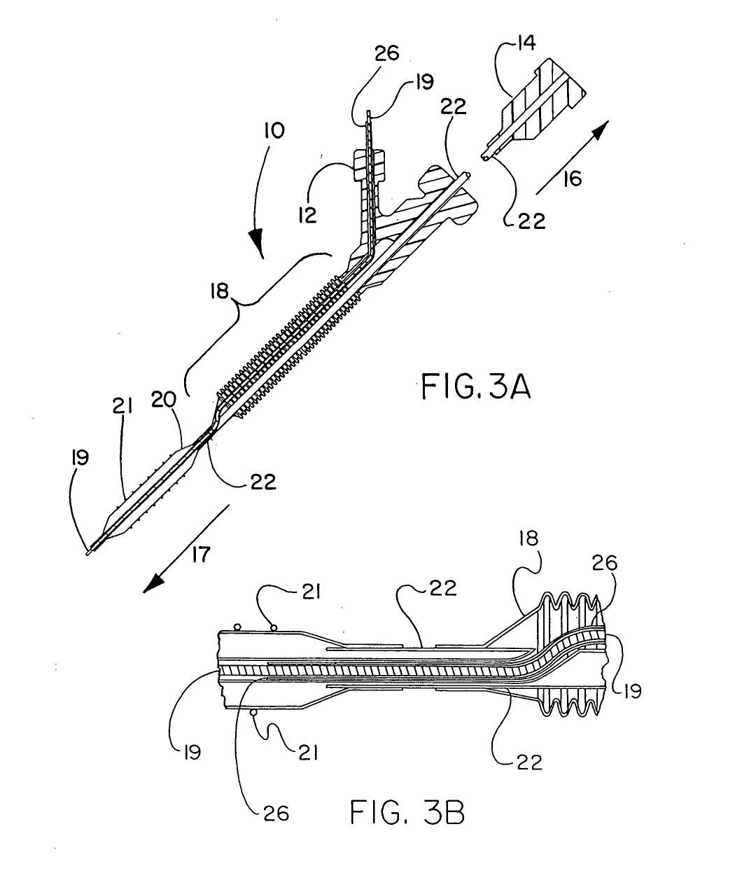

FIG. 3A describes a longitudinal cross section of a catheter 10 similar to the catheter of FIG. 1A with the addition of a threading tube 26 coaxial with guidewire 19. An enlargement of the portion of catheter 10 located between the proximal end of balloon 20 and the distal end of the adjustable length guidewire catheter lumen 18 is described by the longitudinal cross section of FIG. 3B. With the adjustable length guidewire catheter lumen 18 in the compressed or shortened configuration as shown by FIG. 3A, the threading tube 26 may be coaxial with the inflation lumen 22 for a portion of the length of threading tube 26 and adjacent to the inflation lumen 22 for the remainder of its length. With adjustable length guidewire catheter lumen 18 axially compressed, a guidewire 19 may need to be threaded from the distal tip of the catheter 10 and entirely through the guidewire lumen to exit the side arm of y-fitting 12. Threading tube 26 assists in directing guidewire 19 through the adjustable length portion 18 of catheter 10, such that the proximal tip of guidewire 19 does not catch on the corrugations of the shortened adjustable length lumen 18. As shown by FIG. 3A, threading tube 26 extends from the location in the catheter where the guidewire and inflation lumens transition proximally from coaxial to adjacent relationships, and continues to extend beyond where guidewire 19 exits the proximal end 16 of y-fitting 12. This tube 26 preferably has an inside diameter slightly larger than outside diameter of the guidewire 19. For example, for use with a guidewire 19 having an outside diameter of 0.36 mm, a desirable threading tube 26 might have a 0.37 mm inside diameter. A suitable tube is a 0.37 mm inside diameter polyimide tube with a 0.03 mm nominal wall thickness (part number 145, MicroLumen, Tampa, Fla.). If the distal section of the guidewire lumen has a minimum 0.43 mm inside diameter, this polyimide threading tube 26 may be inserted though the entire guidewire path, from the distal tip of the catheter, through the balloon 20 and adjustable length guidewire catheter lumen 18, to the side arm of the slideable hemostasis y-fitting 12. This fixed length threading tube 26 may either be permanently bonded to the distal section of the guidewire lumen, for example by a thermal process or use of an adhesive, or may be fixed by friction alone. It allows guidewire 19 to be easily threaded from the distal tip of the catheter 10 to exit the side arm of y-fitting 12 when the adjustable length lumen 18 is axially compressed to its fully shortened state. After initially threading guidewire 19, if threading tube 26 is removable, it may be removed and discarded. Alternatively, if the distal end of threading tube 26 is permanently fixed, its proximal end will be advanced completely within the adjustable length lumen 18 when adjustable length lumen 18 is extended. When the adjustable length lumen 18 is again shortened by axial compression, guidewire 19 will rethread threading tube 26 back into the side arm of y-fitting 12.

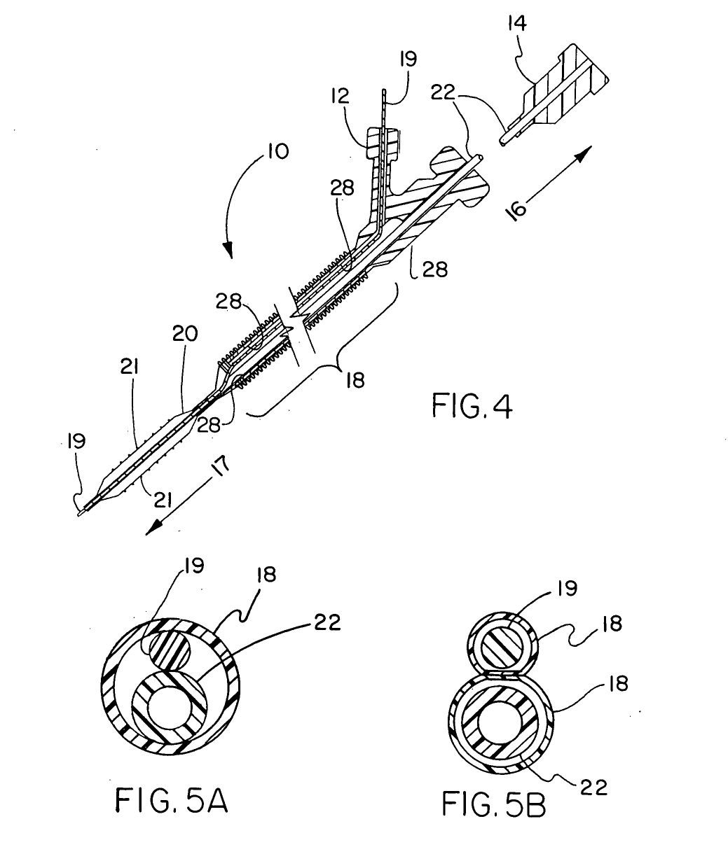

FIG. 4 illustrates a longitudinal cross section of a catheter 10 having a y-fitting 12 that includes a tubular extension 28 at its distal end that protects the compressed adjustable length guidewire catheter lumen 18 (shown in its axially compressed or shortened state) from guidewire 19. This tubular extension 28 may be made as an integral part of y-fitting 12 or may be separately attached to y-fitting 12. This tubular extension 28 facilitates threading of the guidewire. When the distal portion of the catheter is advanced, this tubular extension distances itself from the distal portion of the catheter and does not interfere with the flexibility of the distal portion of the catheter.

FIGS. 5A and 5B show alternative transverse cross sections of the adjustable length guidewire catheter lumen 18. FIG. 5A describes a preferred embodiment wherein guidewire 19 and inflation lumen 22 run collaterally within the adjustable length lumen 18. Alternatively as shown by FIG. 5B, the adjustable length lumen 18 may provide individual lumens in side-by-side relationship for guidewire 19 and inflation lumen 22. Both embodiments may use either pre-formed corrugations or alternatively may be allowed to corrugate non-uniformly under compression.

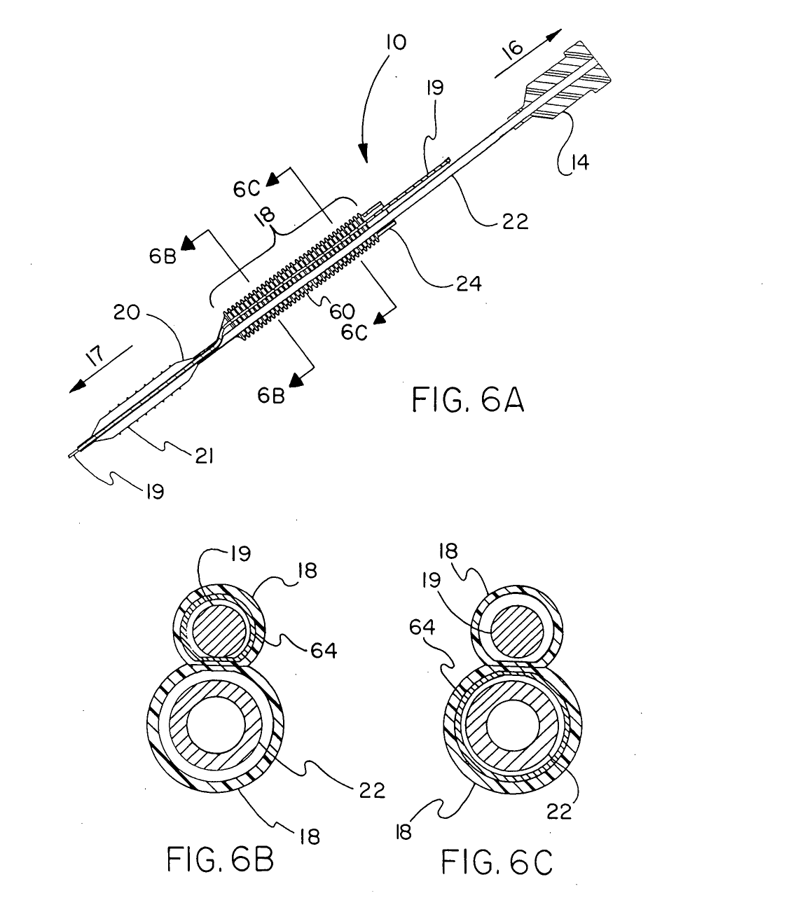

FIG. 6A shows a longitudinal cross section of a catheter guidewire having two different length portions that are separately coated with high-friction coatings that allows the operator to grip the catheter and any component using digital pressure within the coated portion to prevent respective axial movement relative to the gripping hand. FIGS. 6B and 6C show transverse cross sections of different portions of the catheter of FIG. 6A. The entire length of catheter 10, proximal of the distally-positioned balloon 20 may incorporate an adjustable length lumen 18 on its exterior. The ends of this adjustable length lumen 18 are preferably fixed both proximally and distally relative to the remainder of the catheter 10. In the embodiment illustrated, the adjustable length lumen has a ‘figure eight’ cross section; contained in one of the lumens is the inflation lumen 22 and contained in the other lumen is the guidewire 19. A visible marker 60 delineates the distal to the proximal lengths of the adjustable length lumen 18. On the distal length (FIG. 6B), the guidewire lumen is internally coated with a high coefficient of friction material 64 (e.g., silicone), while on the proximal length (FIG. 6C) the other lumen (i.e. containing the inflation lumen) is internally coated with high friction material 64. The physician can then, using both hands, grip the catheter and wire on opposite sides of the visible marker 60. By applying digital pressure through the adjustable length lumen 18, the physician can move the inflation lumen 22 relative to the guidewire 19 to advance or retract the catheter. The catheter can be designed of sufficient length to fully contain the proximal portion of the guidewire during the catheter's operation, minimizing the risk of infecting the guidewire.

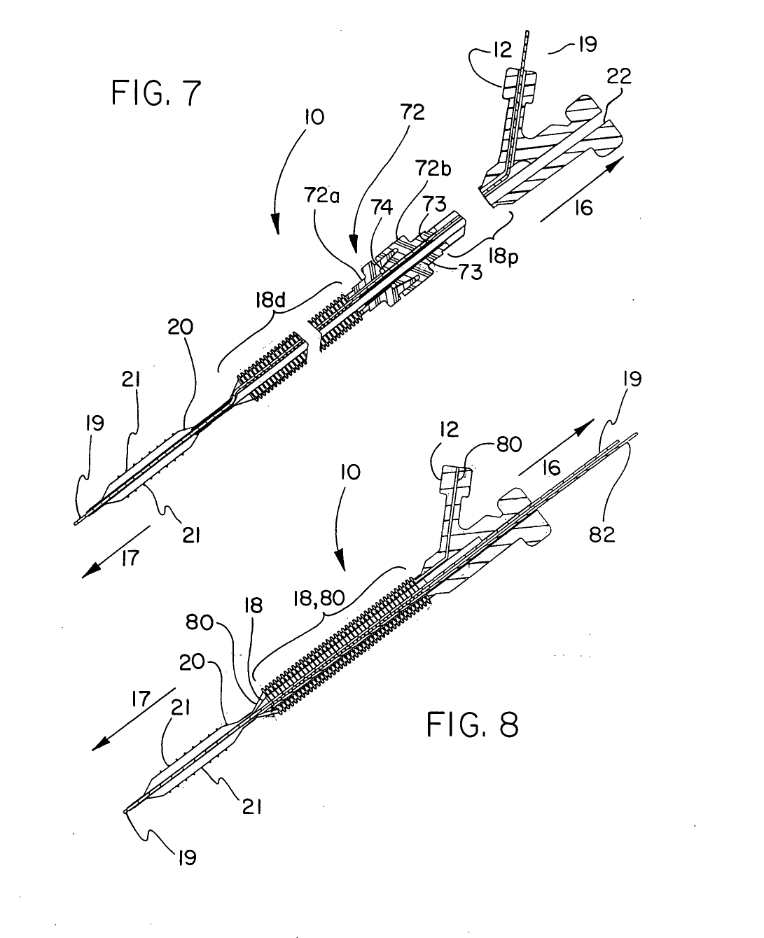

FIG. 7 describes a longitudinal cross section of a catheter having two adjustable length guidewire catheter lumens 18d and 18p (the distal adjustable length guidewire catheter lumen 18d shown in its axially compressed or shortened state while the proximal adjustable length guidewire catheter lumen 18p is shown in its fully lengthened state), separated by a guidewire gripping component 72 positioned about the exterior of the guidewire 19 and inflation lumen 22. In use, when the distal adjustable length guidewire catheter lumen 18d is in a compressed state, the proximal adjustable length guidewire catheter lumen 18p will be extended, and vice versa. Gripping component 72 may be actuated to grip guidewire 19 independent of the inflation lumen 22. Within the gripping component 72 is a rigid hypotube 73 around the inflation lumen; this hypotube 73 is affixed to the lumen of one side of gripping component 72. Gripping component 72 may be actuated to grip any location along the proximal length of the catheter to compress an elastomeric o-ring to fix the guidewire 19 against the outer surface of hypotube 73. It is designed such that when actuated, it grips guidewire 19 but allows the hypotube 73 to slide freely over the inflation lumen 22. Because the adjustable length guidewire catheter lumens 18d and 18p are thin, and because the outside diameter of inflation lumen 22 (e.g., approximately 1.0 mm) is significantly larger than the diameter of the guidewire 19 (e.g., approximately 0.4 mm), the inflation lumen 22 is easily gripped through the walls of either of the adjustable length guidewire catheter lumens 18d and 18p.

Gripping component 72 may be made in various ways to provide the desired gripping action. FIG. 7 shows one construction wherein gripping component 72 has two ends 72a and 72b that may be compressed together against elastomeric o-ring 74, thereby compressing o-ring 74 and forcing it to grip guidewire 19. Alternatively, gripping component 72 may be an in-line hemostasis valve (e.g., an in-line Touhy Borst fitting (e.g., P/N 80352 available from Qosina, Edgewood, N.Y.)).

FIG. 8 describes a longitudinal cross section of a catheter 10 having an adjustable length guidewire catheter lumen 18 located within an adjustable length inflation lumen 80 (with both of these lumens 18 and 80 shown in their axially compressed or shortened states). The length of both of these lumens 18 and 80 is changed by the use of extending means such as a wire 82 that may be pushed or pulled. Adjustable length inflation lumen 80 is in fluid communication with balloon 20 on the distal end 17 of the catheter 10. As illustrated, this embodiment is similar to conventional over-the-wire systems, differing in that both the inner inflation lumen 80 and the outer guidewire lumen 18 are adjustable in length. Pusher wire 82 is used to control advancement and retraction of the distal tip of the catheter 10. Prior to inflation of the balloon, a hemostasis valve 12a on the proximal end of the y-fitting 12 should be closed to ensure minimal leakage from this proximal port. Optionally, instead of pusher wire 82, internal lumen pressure could be used to advance the tip of the catheter 10; however, the use of pusher wire 82 is anticipated to offer better control.

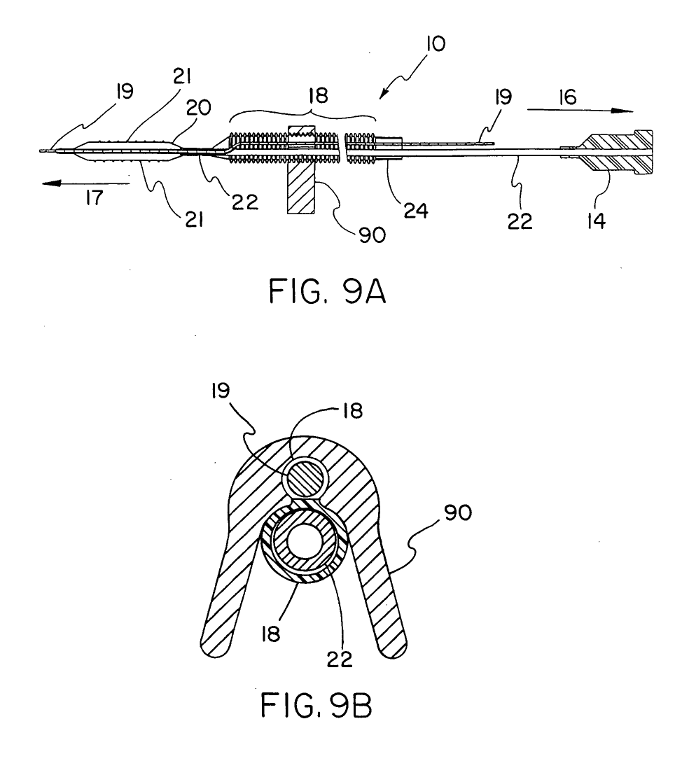

FIG. 9A shows a longitudinal cross section of a catheter having an adjustable length guidewire catheter lumen with guidewire clamp 90. FIG. 9B shows a transverse cross section of the guidewire clamp 90 of FIG. 9A in use on the catheter 10. Clamp 90 may be squeezed by the medical practitioner to grip guidewire 19, allowing for precise movement of the guidewire 19 with respect to the inflation lumen.

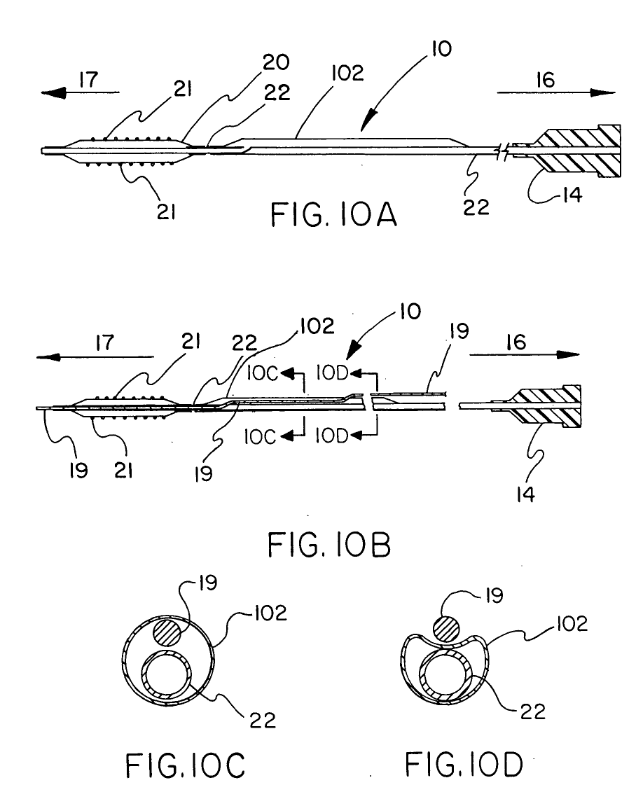

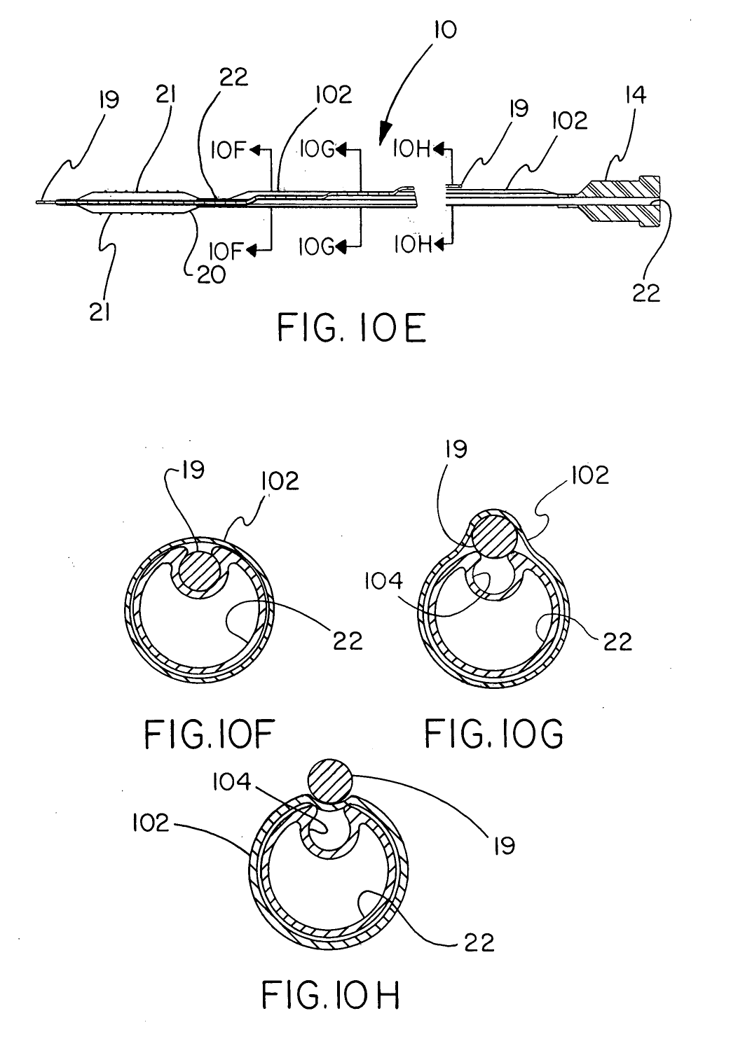

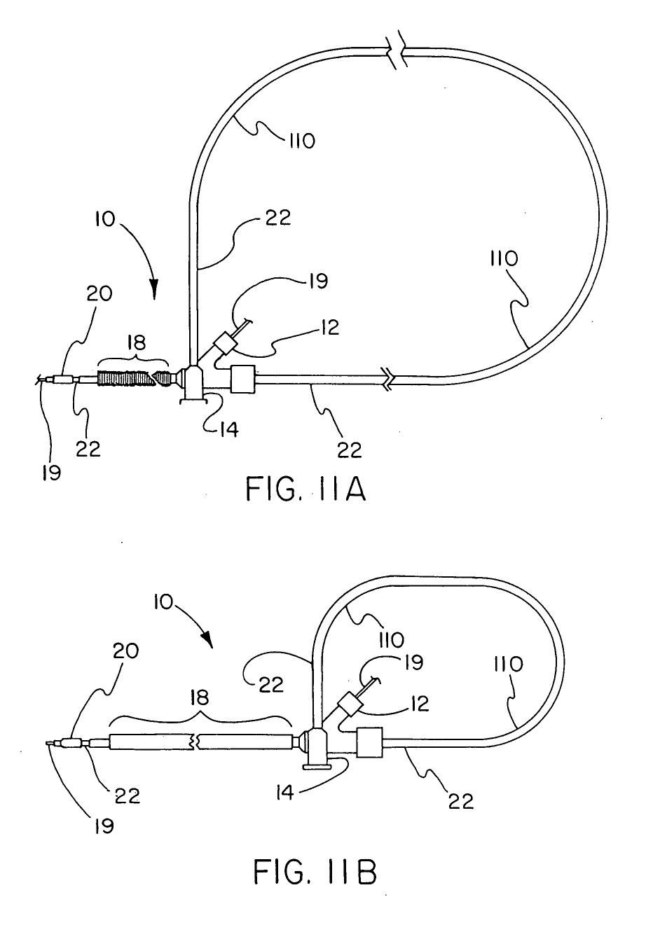

As shown by FIGS. 10A-10H, a thin-walled coaxial lumen 102, designed to be perforated by the proximal tip of a guidewire may be placed coaxially about the inflation lumen 22. After feeding guidewire 19 through the distal section of the guidewire lumen and into the thin-walled coaxial lumen, the physician may chose any desired location along the length of thin-walled lumen 102 at which to perforate lumen 102 with the guidewire 19. In this fashion the physician may select his preferred length of the guidewire lumen.

FIG. 10A shows a longitudinal cross section of a catheter 10 having a puncturable guidewire lumen covering 102, while FIG. 10B shows a longitudinal cross section of the catheter of FIG. 10A in use with the guidewire 19, the guidewire having punctured the puncturable guidewire lumen covering 102. FIGS. 10C and 10D show, respectively, transverse cross sections of the catheter of FIG. 10B with the guidewire 19 within and outside of the puncturable section 102.

FIG. 10E shows a longitudinal cross section of a catheter that is a variation of the design shown in FIGS. 10A and 10B wherein the guidewire operates in a slot 104 provided in the exterior wall of a lumen of the catheter. It is apparent that the puncturable material 102 may be provided only over this slot portion and is not required to enclose the entire circumference of the inner catheter. FIGS. 10F, 10G and 10H show transverse cross sections taken at three different locations along the length of the catheter shown in FIG. 10E.

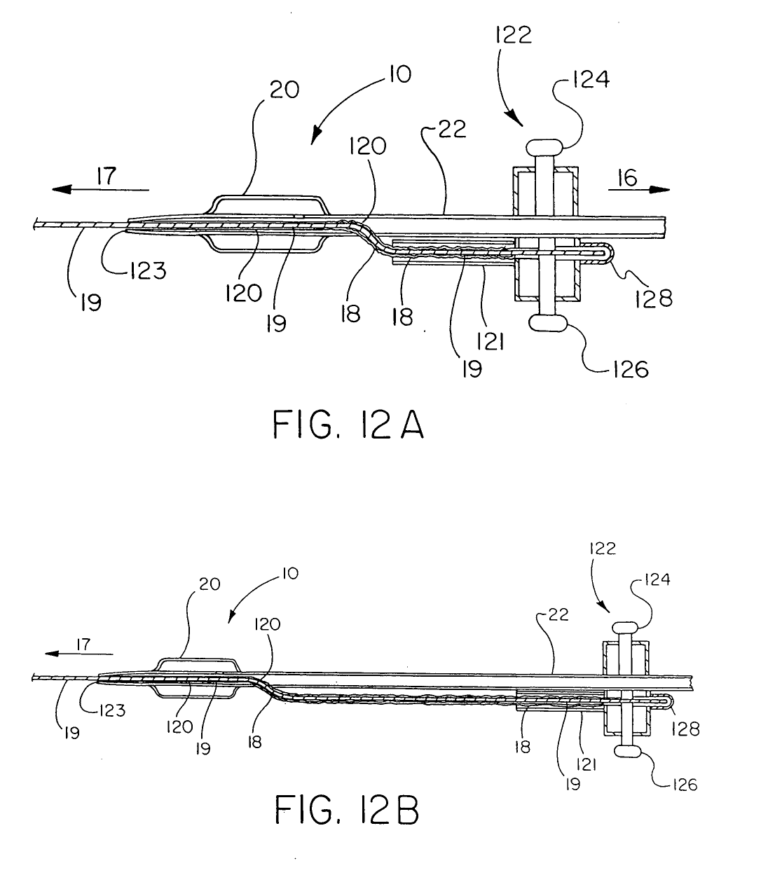

FIG. 11A describes a side view of a catheter 10 having an adjustable length guidewire catheter lumen 18 (shown in its axially compressed or shortened state) with the length of the inflation lumen 22 that extends proximally beyond the y-fitting 12 formed into a loop 110 with the hub 14 of inflation lumen 22 affixed to the y-fitting 12, thereby creating a three exit port fitting. FIG. 11B shows a side view of the catheter 10 of FIG. 11A, except that the adjustable length guidewire catheter lumen 18 is now shown in its axially extended state. The length of the inflation lumen 22 that extends proximally beyond y-fitting 12 formed into a loop 110 that is reduced in length from loop 110 shown in FIG. 11A by the amount of the extension of the adjustable length guidewire catheter lumen 18. The use of loop 110, resulting from attachment of hub 14 to y-fitting 12, provides a simple means of allowing catheter 10 to be operated by a single practitioner. The attachment of these two components results in the creation of a three exit port fitting. This attachment may be accomplished using, for example, a cyanoacrylate adhesive; alternatively, hub 14 and y-fitting 12 may be molded as a unitary, single piece three exit port fitting. Loop 110 is easy to control, and minimizes the risk of the proximal end of the catheter 10 falling from the procedural table. This configuration advantageously allows all functions of the catheter to be controlled at one location, including maintenance of the guidewire position, advancement and retraction of the catheter, inflation and deflation of the balloon, and small adjustments of position of the guide catheter. Additionally, a hemostasis fitting may be attached to the guidewire exit arm of the y-fitting 12 to allow the practitioner to lock the guidewire position relative to the y-fitting 12.

FIGS. 12A and 12B show longitudinal cross sections a balloon catheter with an external adjustable length guidewire catheter lumen 18, wherein FIG. 12A describes the external adjustable length guidewire catheter lumen 18 in an axially compressed, shortened state and FIG. 12B describes the adjustable length guidewire catheter lumen 18 in the fully extended, lengthened state. A guidewire lumen extends continuously through the interior of balloon 20, exits the inflation lumen 22 and extends exterior to the inflation lumen 22 to terminate in the fixation clip assembly 122. The guidewire lumen exterior to the inflation lumen 22 is an adjustable length guidewire catheter lumen 18, extending to seal 128 on the proximal end. The guidewire lumen distal of the adjustable length portion 18 (extending through balloon 20 to the guidewire distal port 123) is a fixed length portion 120.

In use, the guidewire 19 is inserted into the distal guidewire port and through the continuous guidewire lumen until the proximal tip of guidewire 19 is located just distal to the fixation clip assembly 122. Guidewire clamp 126 is then released by squeezing manually, and the guidewire 19 further inserted into the seal 128 located on the proximal end of the guidewire lumen. The guidewire clamp 126 is then released to secure the guidewire 19 to the fixation clip assembly 122. Preferably, at least a portion of the fixation clip assembly 122 is transparent to allow verification of the position of guidewire 19. In addition, seal 128 is preferably made from a compliant material such as silicone to allow manual gripping of guidewire 19. The fixation clamp assembly 122 has an integral support sleeve 121 to contain the adjustable length guidewire catheter lumen 18 when in its axially compressed, shortened state. Support sleeve 121 encourages guidewire 19 to follow the guidewire lumen during insertion, and also contains the axially compressed adjustable length guidewire catheter lumen 18 during shipment.

The balloon 20 is progressed into and through the vasculature by squeezing the inflation lumen clamp 124 with one hand while advancing the inflation lumen 22 with the other hand. As balloon 20 is advanced, the adjustable length guidewire catheter lumen 18 becomes extended, as shown in FIG. 12B. When the balloon 20 is positioned at the desired site, the inflation lumen clamp 124 is released, securing the location of guidewire 19 relative to the inflation lumen 22. The fixation clamp assembly 122 can then be secured relative to the patient if desired, freeing both hands of the practitioner for inflation of balloon 20.

The fixed length portion 120 of the guidewire lumen (distal of the adjustable length portion 18) is preferably a tube of non-porous PTFE, while adjustable length guidewire catheter lumen 18 is preferably made from ePTFE as described previously. The tube of the fixed length portion 120 can be inserted into an appropriately sized hole in the wall of inflation lumen 22, proximal to balloon 20 as shown. The fixed length portion tube 120 can then be sealed to the catheter distal tip and to the hole in the inflation lumen wall.

### EXAMPLE

A catheter was constructed using a very thin walled (e.g., 0.03 mm) sheath material. The sheath material is required to be thin enough to corrugate in small folds, allowing the length of the sheath to be reduced to less than 50% of its original length by compressing into the small amplitude folds. A 0.01 mm thick ePTFE membrane provided with a non-porous FEP coating on one side was chosen for the sheath material. This membrane was slit to a 6.4 mm width, thereby forming a tape.

An ePTFE tube, having an inner diameter of about 1.6 mm and a wall thickness of about 0.13 mm, was fitted over a 1.6 mm diameter stainless steel mandrel having a length of about 180 cm. The 6.4 mm wide tape was then helically wrapped about the outer surface of the ePTFE tube with a 50% overlap, resulting in a helically-wrapped tube covered with two layers of tape. The resulting assembly was then placed into an air convection oven set at 320° C. for 8 minutes, after which it was removed from the oven and allowed to cool in an ambient environment.

After cooling, the helically-wrapped tube was removed from the mandrel by withdrawing the mandrel from the tube. The end of the extruded tube that had not been helically-wrapped was clamped in a vise. The end of the helical wrapping closest to the vise was simultaneously pinched on opposite sides of the tube using the thumb and forefingers of both hands, and the helical-wrapping was stripped from the underlying ePTFE tube by everting the helically-wrapped tube while pulling it away from the vise.

This thin-walled tube had an approximate wall thickness of 0.03 mm (measured using Mitutoyo Snap Gauge, Model #1 D-C112EBS) and an inner diameter of approximately 1.7 mm (measured using a certified minus pin gauge with a tolerance of 0.01 mm). When this tube was loaded on a 1.2 mm diameter mandrel, it was able to be easily compressed to about 5% of its original length using light digital pressure.

Continuing assembly of the catheter, this sheath was then coaxially mounted over a conventional Percutaneous Transluminal Coronary Angioplasty (PTCA) catheter with a maximum outer diameter proximal of the balloon of less than approximately 0.040″ (1.02 mm). The PTCA catheter used was a rapid exchange type, having a proximal guidewire exit port at a location significantly distal of its hub. Prior to mounting the sheath, a 9 Fr (3.0 mm) inner diameter hemostasis y-arm valve (P/N 80348, Qosina, Edgewood, N.Y.) was slid onto the catheter from the catheter's distal end (hemostasis valve oriented away from the tip of the catheter). Next, a female luer (P/N 65206. Qosina, Edgewood, N.Y.) was slid onto the catheter and the luer connection of these two components was engaged. A 2.0 mm inside diameter by 2.1 mm outside diameter 304 stainless steel tube (Microgroup, Medway, Mass.) was then swaged down to approximately 1.4 mm inside diameter by 1.6 mm outside diameter, and then trimmed to a length of approximately 19 mm.

This tube was slid coaxially over the catheter and bonded to the distal end of the female luer with an approximate 6 mm overlap using cyanoacrylate adhesive (Loctite 401, Loctite Corp., Rocky Hill, Conn.). Next, the helically-wrapped sheath described above was slid over the distal tip of the catheter and its proximal end attached by sliding it over the exposed end of the hypotube. These overlapped surfaces were bonded using the cyanoacrylate adhesive, after which 2.3 mm inside diameter polyolefin 2-to-1 shrink ratio shrink tubing was fitted over the junction and heated to conform to the surface of the junction. The distal end of the sheath was then trimmed to a length of approximately 135 cm, equal to the desired working length of the catheter (i.e. length from the distal tip of the catheter to the distal end of the strain relief on the catheter's hub). The distal end of the sheath was then attached at a location approximately 2 mm distal of the proximal guidewire port in the wall of the PTCA catheter. This attachment was made using the cyanoacrylate adhesive between the sheath and catheter, and then over-wrapping this attachment point with cyanoacrylate adhesive and 0.13 mm diameter ePTFE suture (CV-8, WL Gore and Associates, Flagstaff, Ariz.).

To complete the catheter a hemostasis y-fitting was slid distally on the catheter until it was just proximal of the proximal hole of the original PTCA catheter. This compressed the sheath to approximately 15% of its original approximately 135 mm length. A guidewire was then fed into the distal tip of the catheter and carefully threaded through the catheter, including the sheath component, and out from the proximal end of the catheter through the side arm of the y-fitting.

With the guidewire inserted, the user was able to hold the guidewire and hemostasis y-fitting in a fixed position while advancing the distal tip of the catheter relative to the guidewire. Compared to a standard catheter with a proximal guidewire side port fixed distally of the proximal hub, this inventive catheter significantly improved the ability of the section of the catheter, distal to the hemostasis y-fitting, to track the guidewire and allow push forces applied to the proximal portion of the catheter shaft to be transferred directly to the distal tip of the catheter.

While the principles of the invention have been made clear in the illustrative embodiments set forth herein, it will be obvious to those skilled in the art to make various modifications to the structure, arrangement, proportion, elements, materials and components used in the practice of the invention. To the extent that these various modifications do not depart from the spirit and scope of the appended claims, they are intended to be encompassed therein.

We claim:

1. A catheter assembly comprising
a pushable element; and
a guidewire catheter lumen positioned collateral with the pushable element, the guidewire catheter lumen formed from a scrunchable material;
whereby at least a portion of the guidewire catheter lumen is adjustable in length by an amount of at least ten percent.

2. A catheter according to claim 1 wherein the guidewire catheter lumen is adjustable in length by an amount of at least twenty percent.

3. A catheter according to claim 1 wherein the guidewire catheter lumen is adjustable in length by an amount of at least thirty percent.

4. A catheter according to claim 1 wherein the guidewire catheter lumen is adjustable in length by an amount of at least fifty percent.

5. A catheter according to claim 1 wherein the guidewire catheter lumen is adjustable in length by an amount of at least seventy five percent.

6. A catheter according to claim 1 wherein the guidewire catheter lumen is adjustable in length by an amount of at least one hundred percent.

7. A catheter according to claim 1 wherein the guidewire catheter lumen is adjustable in length by an amount of at least two hundred percent.

8. A catheter according to claim 1 wherein the guidewire catheter lumen is adjustable in length by an amount of at least four hundred percent.

9. The catheter assembly of claim 1 wherein the scrunchable material comprises a fluoropolymer.

10. The catheter assembly of claim 9 wherein the fluoropolymer includes polytetrafluoroethylene.

11. The catheter assembly of claim 10 wherein the fluoropolymer material includes porous polytetrafluoroethylene.

12. The catheter assembly of claim 1 wherein the scrunchable material comprises a thermoplastic.

13. The catheter assembly of claim 12 wherein the scrunchable material comprises polyethylene terephthalate.

14. The catheter assembly of claim 1 wherein the guidewire catheter lumen includes at least two sections, each section including an adjustable length guidewire catheter portion.

15. The catheter assembly of claim 14 wherein the at least two sections change length in cooperation with one another so as to maintain a substantially constant overall length of the guidewire catheter throughout an overall range of operation.

16. The catheter assembly of claim 1 wherein the pushable element is an inflation tube.

17. The catheter assembly of claim 1 wherein the pushable element is a pushwire.

18. The catheter assembly of claim 1 wherein the scrunchable material has a thickness less than about 0.20 mm.

19. The catheter assembly of claim 1 wherein the scrunchable material is inelastic.

20. The catheter assembly of claim 1 including a balloon.

21. The catheter assembly of claim 1 including guidewire catheter having a wall that includes no aperture open to an exterior of the catheter assembly.

22. The catheter assembly of claim 1 wherein the guidewire catheter provides a channel for a guidewire that provides smooth pushability of the catheter assembly along a guidewire.

23. The catheter assembly of claim 1 wherein the guidewire catheter lumen is adjustable in length via corrugations.

24. The catheter assembly of claim 1 including a threading tube.

25. The catheter assembly of claim 1 including a guidewire.

26. The catheter assembly of claim 1 wherein the guidewire does not pass through an aperture in a wall of the catheter assembly to an exterior of the catheter assembly.

27. The catheter assembly of claim 1 wherein the guidewire does not pass through an aperture in a wall of the adjustable length guidewire catheter lumen to an exterior of the adjustable length guidewire catheter lumen.

28. The catheter assembly of claim 1 wherein the scrunchable material is less than about 0.2 mm thick.

29. The catheter assembly of claim 1 wherein the scrunchable material is a flexible material.

30. The catheter assembly of claim 1 wherein the guidewire catheter lumen is puncturable by a guidewire.

31. A catheter assembly comprising
a pushable element; and
a guidewire catheter lumen positioned collateral with the pushable element, the guidewire catheter lumen formed from a scrunchable material;
a guidewire catheter coextensive with the guidewire catheter lumen, the guidewire catheter and the adjustable length guidewire catheter lumen having walls that includes no aperture open to an exterior of the catheter assembly;
whereby at least a portion of the guidewire catheter lumen is adjustable in length by an amount of at least ten percent.

32. A catheter assembly comprising an inflation lumen having a length;
a slideable y-fitting located along the length between distal and proximal portions of the catheter assembly;
wherein the proximal portion is formed into a loop having a length and terminating at the slideable y-fitting.

33. A catheter assembly according to claim 32 wherein the loop terminates at a hub component affixed to the y-fitting.

34. A catheter assembly according to claim 33 wherein the hub component affixed to the y-fitting comprises a three exit port fitting.

35. A catheter assembly according to claim 32 wherein the distal portion is an adjustable length lumen which when adjusted in length changes the length of the loop.

36. A catheter assembly according to claim 33 wherein the distal portion is an adjustable length lumen which when adjusted in length changes the length of the loop.

37. A catheter assembly according to claim 34 wherein the distal portion is an adjustable length lumen which when adjusted in length changes the length of the loop.
